(19) United States
(12) Patent Application Publication
(10) Pub. No.: US 2008/0167718 A1
(43) Pub. Date: Jul. 10, 2008

(54) Active Vertebral Prosthetic Device

(75) Inventor: Dimitri K. Protopsaltis, Memphis, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/620,280

(22) Filed: Jan. 5, 2007

(51) Int. Cl. A61F 2/44

(52) U.S. Cl. 623/17.16; 623/17.11; 623/17.15

(57) ABSTRACT

An active vertebral prosthetic device system may include an actuatable displacement element having an upper side and a lower side. The actuatable displacement element may be configured for placement between an upper vertebral body and a lower vertebral body and may be configured to alter the overall distance between the upper and the lower vertebral bodies in situ. A controller may be operable to post-surgically actuate the actuatable displacement element. In some aspects, the displacement element maybe a piezoelectric motor, an electroactive polymer, an ionic polymer metal composite, and an actuator.

This application is related to co-pending U.S. patent application Ser. No. ______, titled Non-Rigid Intervertebral Spacers, having the same filing date as the present application, (Attorney Docket No. P26223/31132.626), incorporated herein in its entirety by reference

### BACKGROUND

Disc arthroplasty is one way of treating injured, degraded, or diseased spinal discs. Some disc arthroplasty treatments include replacing injured discs of the joint with either a fused or a motion-preserving spinal disc that replaces the injured disc at the spinal joint. However, after implantation, there may be an occasional need to adjust the spinal disc. For example, it is possible that the disc may not have been properly located by the operating physician. It is also possible that post-operative movement may occur prior to fusion or bonding with the associated vertebrae.

In such instances, it is often not desirable to perform another surgery to correct the position of the disc. Inserting the spinal disc can be an invasive and intensive procedure. For example, anterior procedures often require displacement of organs, such as the aorta and vena cava, and must be performed with great care. Further, because scar tissue may grow about the surgical site, any required second treatment can be more difficult, and may introduce additional distress to the patient.

In addition, due to either disc design or placement, the disc may not provide support that properly mimics a natural disc. For example, fusion discs eliminate movement at the spinal joint, while articulating discs may not match the movement that occurs at a natural disc.

### SUMMARY OF THE INVENTION

In one exemplary aspect, this disclosure is directed to an active vertebral prosthetic device system. The system may include an actuatable displacement element having an upper side and a lower side. The actuatable displacement element may be configured for placement between an upper vertebral body and a lower vertebral body and may be configured to alter the overall distance between the upper and the lower vertebral bodies in situ. A controller may be operable to post-surgically actuate the actuatable displacement element.

In another exemplary aspect, this disclosure is directed to an active vertebral prosthetic device system including a first endplate configured to cooperatively engage an upper vertebral body and a second endplate configured to cooperatively engage a lower vertebral body. An actuatable displacement element may be operably disposed between the first and the second endplates. The actuatable displacement element may be configured to change the overall distance between the first endplate and the second endplate in situ. A controller may be operable to post-surgically actuate the actuatable displacement element.

In yet another exemplary aspect, this disclosure is directed to a vertebral prosthetic device system for implantation in a body. The system may include an active vertebral prosthetic device including an actuatable displacement element having an upper side and a lower side. The active vertebral prosthetic device may be configured for placement between and in contact with an upper vertebral body and a lower vertebral body and may be configured to alter the overall distance between the upper and the lower vertebral bodies in situ. A controller may be operable to post-surgically communicate with the active vertebral prosthetic device. A sensor may be configured for implantation in the body and may be in communication with the controller. The sensor may be configured to provide data to the controller and the controller may be configured to process the data and control the actuatable displacement element based on the processed data.

In yet another exemplary aspect, this disclosure is directed to a method including the steps of implanting an actuatable displacement element between an upper vertebral body and a lower vertebral body. The actuatable displacement element may have an upper side and a lower side respectively facing the upper vertebral body and the lower vertebral body. The method also may include post-surgically actuating the actuatable displacement element with a controller to alter the overall distance between the upper and lower vertebral bodies.

### BRIEF DESCRIPTION OF THE DRAWINGS

FIG. 1 is an illustration of a lateral view of a portion of a vertebral column.

FIG. 2 is an illustration of a lateral view of a pair of adjacent vertebral bodies defining an intervertebral space.

FIG. 3 is an illustration of an exemplary intervertebral prosthetic device disposed between adjacent vertebral bodies.

FIG. 4 is an illustration of a portion of another exemplary intervertebral prosthetic device.

FIG. 5 is an illustration of the intervertebral prosthetic device of FIG. 3.

FIG. 6 is an illustration of the intervertebral prosthetic device of FIG. 4 in a tilted position.

FIG. 7 is an illustration of a portion of yet another exemplary intervertebral prosthetic device.

FIG. 8 is an illustration of another exemplary intervertebral prosthetic device.

FIG. 9 is an illustration of another exemplary intervertebral prosthetic device.

FIG. 10 is an illustration of a cross-section of another exemplary intervertebral prosthetic device.

FIG. 11 is an illustration of a system incorporating an exemplary intervertebral prosthetic device.

### DETAILED DESCRIPTION

The present invention relates generally to vertebral reconstructive devices and, more particularly, to an intervertebral prosthetic device for implantation. For the purposes of promoting an understanding of the principles of the invention, reference will now be made to embodiments or examples illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

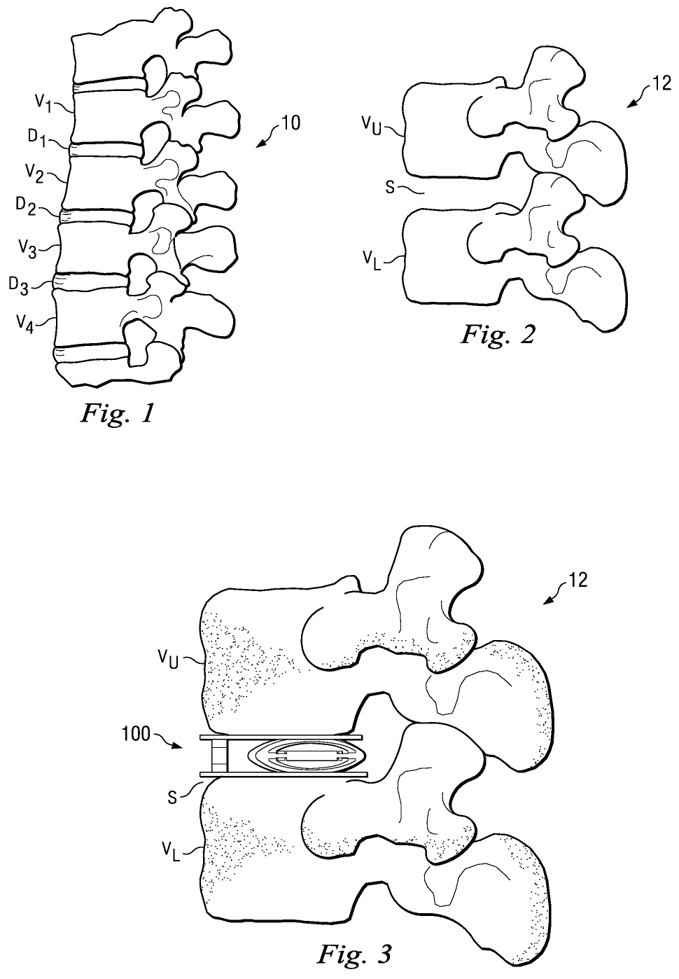

FIG. 1 shows a lateral view of a portion of a spinal column 10, illustrating a group of adjacent upper and lower vertebrae V1, V2, V3, V4 separated by natural intervertebral discs D1, D2, D3. The illustration of four vertebrae is only intended as an example. Another example would be a sacrum and one vertebra.

For the sake of further example, two of the vertebrae will be discussed with reference to a spinal segment 12 shown in FIG. 2. The two vertebrae form a spinal segment including an upper vertebrae VU and a lower vertebrae VL. Some types of disc arthroplasty require that some or all of the natural disc that would have been positioned between the two vertebrae VU, VL be removed via a discectomy or a similar surgical procedure. Removal of the diseased or degenerated disc results in the formation of an intervertebral space S between the upper and lower vertebrae VU, VL. Although the illustration of FIG. 2 generally depicts the vertebral joint as a lumbar vertebral joint, it is understood that the devices, systems, and methods of this disclosure may also be applied to all regions of the vertebral column, including the cervical and thoracic regions.

FIG. 3 shows a side view of the vertebrae VU, VL with an intervertebral prosthetic device 100 in the disc space S. Sized to fit the disc space height in a manner similar to a natural intervertebral disc, such as any of discs D1-D4, the prosthetic device 100 provides support and stabilization to the vertebrae. However, the prosthetic device 100 also allows the vertebra VU to more relative to the vertebra VL to provide movement or articulation to the spinal joint. In the embodiment shown, the prosthetic device 100 is an active prosthetic device capable of increasing or decreasing in height, as well as tilting or changing the pitch in either the sagittal, axial, or coronal planes. The prosthetic device 100 includes actuatable displacement elements that allow for movement, articulation, and other displacement at the disc joint.

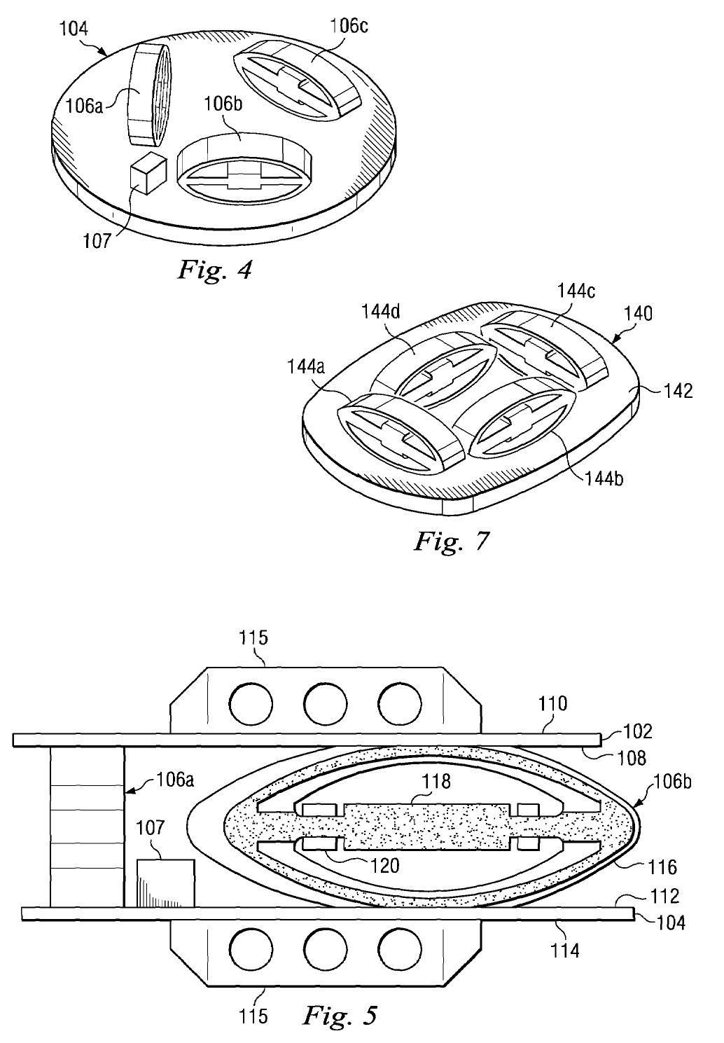

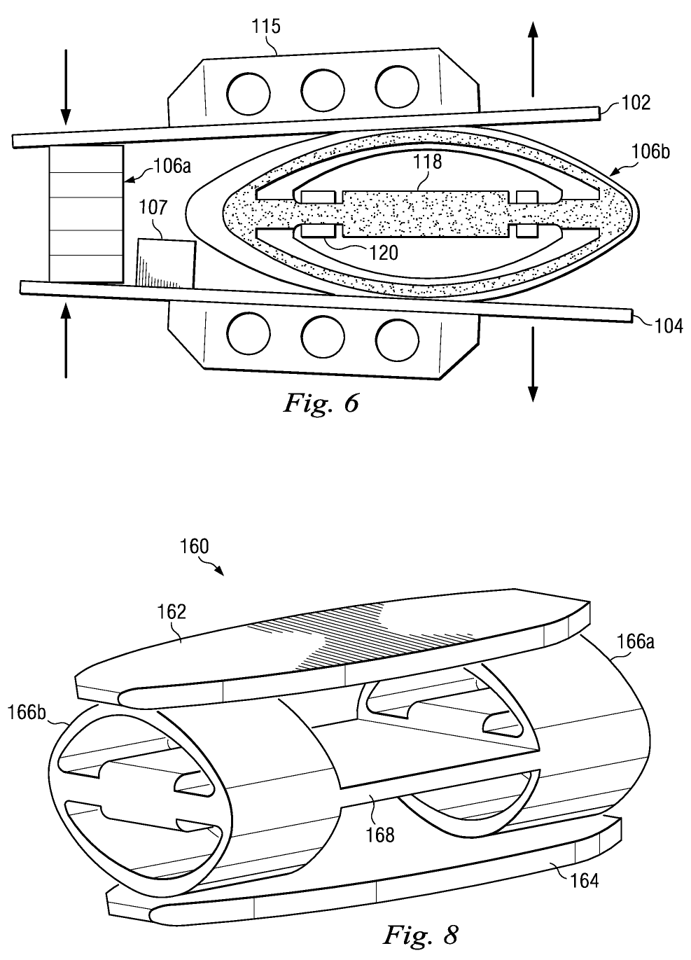

FIGS. 4-6 show the prosthetic device 100 in greater detail. FIG. 4 shows a perspective view of the prosthetic device 100 having one endplate removed. FIG. 5 shows a side view of the prosthetic device 100 in a neutral position, and FIG. 6 shows the prosthetic device 100 in a tilted or pitched configuration. Referring now to FIGS. 4-6, the prosthetic device 100 includes an upper endplate 102 (shown in FIGS. 5 and 6), a lower endplate 104, and three displacement elements 106a, 106b, and 106c. In FIGS. 5 and 6, showing side views, only the displacement elements 106a and 106b are visible. Some embodiments include a controller 107 operable to actuate the displacement elements 106.

As best seen in FIGS. 5 and 6, the upper endplate 102 includes an inner surface 108 and an outer surface 110. The inner surface 108 may be configured to cooperate with the displacement elements 106a, 106b, and the outer surface 110 may be configured to cooperatively engage a bone structure such as the upper vertebral body VU of FIG. 3, either directly or through additional components, such as, for example, additional endplates or cages.

Similar to the upper endplate 102, the lower endplate 104 includes an inner surface 112 and an outer surface 114. The inner surface 112 may be configured to cooperate with the displacement elements 106a, 106b, and the outer surface 114 may be configured to cooperatively engage a bone structure such as the lower vertebral body VL of FIG. 3, either directly or through additional components, such as, for example, additional endplates or cages.

The upper and lower endplates 102, 104 may be formed of any suitable biocompatible material including metals such as cobalt-chromium alloys, titanium alloys, nickel titanium alloys, and/or stainless steel alloys. Ceramic materials such as aluminum oxide or alumina, zirconium oxide or zirconia, compact of particulate diamond, and/or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Other suitable materials also may be used. Furthermore, the inner and outer surfaces of one or both of the endplates need not be parallel, but may be angled relative to each other.

The outer surfaces 110, 114 of the upper and lower endplates 102, 104, in embodiments where they directly contact bone, may include features or coatings which enhance the fixation of the prosthetic device 100. For example, the outer surfaces 110, 114 may be roughened such as by chemical etching, bead-blasting, sanding, grinding, seriating, and/or diamond-cutting. All or a portion of the outer surfaces 110, 114 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, and/or other surface textures.

In the exemplary embodiment shown in FIGS. 5 and 6, optional upper and lower bone connectors 115 are formed on the outer surfaces 110, 114. These bone connectors 115 extend toward the upper and lower vertebrae in a manner to help secure the prosthetic device 100 in place. In the example shown, the bone connectors 115 are keels configured to extend into notches or grooves formed into the vertebral endplates. Although shown as extending along a substantial length of the upper and lower endplates 102, 104, the bone connectors 115 may be of any length, either shorter or longer than that shown, and in addition, may have some other orientation or features other than that shown. For example, in some embodiments, the bone connectors are a series of ridges, protrusions, or other surface features that help fix the prosthetic device 100 in place. In some embodiments, the bone connector may include a connecting aperture (not shown) configured to receive a bone fastener, such as a screw.

The exemplary displacement elements 106a, 106b in the embodiment in FIG. 5 are piezoelectric tilt motors configured to provide stability and dynamic movement to the prosthetic device 100. The displacement element 106a is shown in an end view, and the displacement element 106b is shown substantially from a side view. The exemplary motors include an outer band 116, a center piston 118, and contact leads 120. The outer band 116 may be formed of a biocompatible material, and may be formed of any of the materials described above with reference to the upper and lower endplates. In the embodiment disclosed, the outer band 116 is formed in an oval shape, with the minor axis intersecting the upper and lower endplates and the major axis being substantially parallel to the upper and lower endplates when in a neutral or beginning position. The outer band 116 may be attached to the endplates 102, 104 in any known manner including, for example, using an adhesive, polymer bonding, welding, and melting, among others. In some embodiments, the endplates and outer bands 116 are integrally formed or machined.

The center piston 118 connects to and extends across a diameter of the outer band 116, and in this case, along the major axis of the oval shaped outer band 116. Piezo electric crystals embedded in or forming a part of the center piston 118 enable it to expand or retract in response to an applied electrical current. The contact leads 120 provide a point of contact for conductors (not shown), such as wires, that provide the electrical current to drive the center piston 118. In the embodiment shown, the center piston 118 lies substantially parallel to the upper and lower endplates 102, 104.

In use, the electrical current causes the center piston 118 to expand or retract, thereby increasing or decreasing the diameter of the outer band along the major axis. As the major axis diameter decreases, the minor axis diameter increases, thereby increasing the distance between the upper and lower endplates 102, 104. Likewise, as the major axis diameter increases, the minor axis diameter decreases. This decreases the distance between the upper and lower endplates 102, 104.

The controller 107 is configured to control actuation of the actuatable displacement elements 106a, 106b. It may do this by controlling the timing of electrical signals sent to the actuatable elements and may generate those signals itself. The controller 107 may be disposed onboard the implantable device 100 and is shown attached to the inner surface of the lower endplate 104. However, in other embodiments the controller 107 may be disposed at other locations onboard the prosthetic device or alternatively, at a location spaced from the prosthetic device. For example, in some embodiments, the controller is disposed as a part of or connected to the center piston 118 within the piezoelectric tilt motors. In other embodiments, the controller is not implanted, but is disposed outside the patient's body. The controller 107 may be configured to generate a signal to control the displacement members of the prosthetic device 100. In some embodiments, the controller 107 includes a processor for processing data, including signals instructing the controller to actuate the displacement elements of the prosthetic device.

FIG. 6 shows the prosthetic device 100 in a tilted or pitched position. In FIG. 6, the displacement element 106a is shown having a decreased height compared to FIG. 5, and the displacement element 106b is shown having an increased height compared to FIG. 5. This ultimately causes the endplates to be non-parallel, giving tilt to the prosthetic device 100. As would be apparent to one skilled in the art, if both the displacement elements 106a, 106b are manipulated to have an equal increased height or an equal decreased height, the overall height of the prosthetic device 100 is correspondingly increased or decreased.

In FIG. 4, the orientation of the three displacement elements 106a, 106b, and 106c allows the endplates 102, 104 of the prosthetic device 100 to tilt in any direction and to change the overall height of the implantable device, thereby changing the distance between the vertebral bodies. The displacement elements are symmetrically arranged about a sagittal plane, simplifying control of the tilt directions.

FIG. 7 shows another exemplary prosthetic device 140. The prosthetic device 140 includes a lower endplate 142, four displacement elements 144a-d, and an upper endplate (not shown). The prosthetic device 140 is arranged and operates in manner similar to that of the prosthetic device 100 described above with reference to FIGS. 3-6. However, rather than three displacement elements, the prosthetic device 140 includes four displacement elements 144a-d arranged symmetrically relative to a sagittal plane. As with the prosthetic device 100 discussed above, the displacement elements 144a-d may be controlled to operate together to increase or decrease the overall height of the implant 140 to increase or decrease the distance between the vertebral bodies, or alternatively, may be controlled to provide tilt in any direction including both the sagittal and coronal planes.

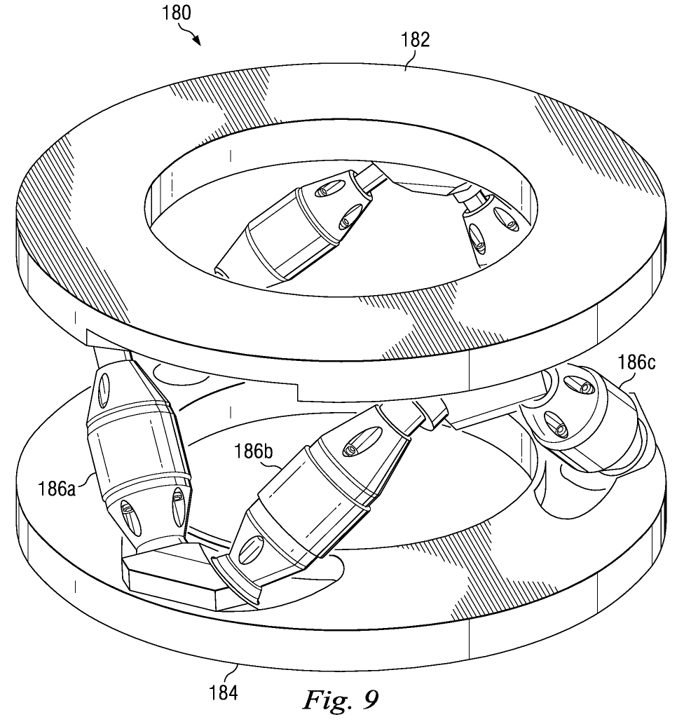

FIG. 8 shows another embodiment of an exemplary prosthetic device 160. The prosthetic device 160 includes an upper endplate 162, a lower endplate 164, and only two displacement elements 166a, 166b. The endplates 162, 164 and displacement elements 166a, 166b may be as those described above with reference to FIGS. 3-6. In addition, the prosthetic device 160 includes a connecting rod 168 extending from one displacement element 166a to the other 166b. The connecting rod 168 provides support and increases the stability of the prosthetic device 160. A controller as described above may be connected to the connecting rod 168, the endplates, or elsewhere.

FIG. 9 shows another embodiment of an exemplary prosthetic device 180. The prosthetic device 180 includes an upper endplate 182, a lower endplate 184, and six displacement elements 186a-f. The upper endplate 182 includes an inner surface 188 and an outer surface 190, and the lower endplate includes an inner surface 192 and an outer surface 194. In this embodiment, the displacement elements 186 are piezoelectric linear actuators arranged in a hexapod. Each of the linear actuators are arranged to extend in a non-perpendicular direction from the inner surface 188 of the upper endplate 182 to the inner surface 192 of the lower endplate 184. In some embodiments, the linear actuators may be arranged to be angled in the range of 10-80 degrees relative to the inner surfaces 188, 192 of the endplates 182, 184. In other embodiments, the linear actuators are arranged to be angled in the range of 20-70 degrees relative to the endplate inner surfaces 188, 192.

The angled orientation of the displacement elements 186 provide multi-level control and allows the upper endplate 102 to be not only tilted relative to the lower endplate 184, but also allows the upper endplate to be moved sideways or fore or aft while maintaining a desired tilt. For example, if a physician were to determine that the top plate should be moved toward the anterior region, the adjustment can be made without changing the desired tilt. Accordingly, the center point of the prosthetic device 180 may adjusted by moving one endplate transversely relative to the other.

While the displacement elements 186 are shown as tilted linear actuators in FIG. 9, it should be noted that the linear actuators may be oriented in a substantially perpendicular direction relative to the inner surfaces of the endplates. This may provide tilt capability as described above relative to the embodiments in FIGS. 3-8. A controller (not shown) also may be included.

While the prosthetic devices have been described as employing piezoelectric motors or piezoelectric actuators as the displacement elements, it should be noted that the displacement elements may be otherwise configured. For example, in some embodiments, the displacement elements do not include a center piston as disclosed in FIGS. 3-6, but instead the outer ring is formed of a piezo electric material that allows the outer ring to actuate to change its diameter. In these embodiments, the outer ring may be formed of a polymer having piezoelectric material embedded therein. In yet other embodiments, the displacement elements are formed of artificial muscles comprised of electroactive polymers (EAP) that actuate in response to electrical current. In these embodiments, the outer ring or the inner piston may be formed of electroactive polymers. In yet other embodiments, the displacement elements may be formed of ionic polymer-metal composites (IPMC). In these embodiments, the displacement elements alter the distance between the inner and outer plates by voltage switching. The voltage switching causes ions and water to inter the IPMC, which causes it to increase its size, driving the endplates apart. In yet other embodiments, the displacement elements are formed of traveling wave actuators.

In addition, it should be noted that some embodiments include only a single displacement element, while others include two, three, four, five, or more. In some embodiments employing a single displacement element, the prosthetic device may be configured to only increase or decrease its height, while in others, it may adjust tilt, such as, for example, when the displacement element is off-center.

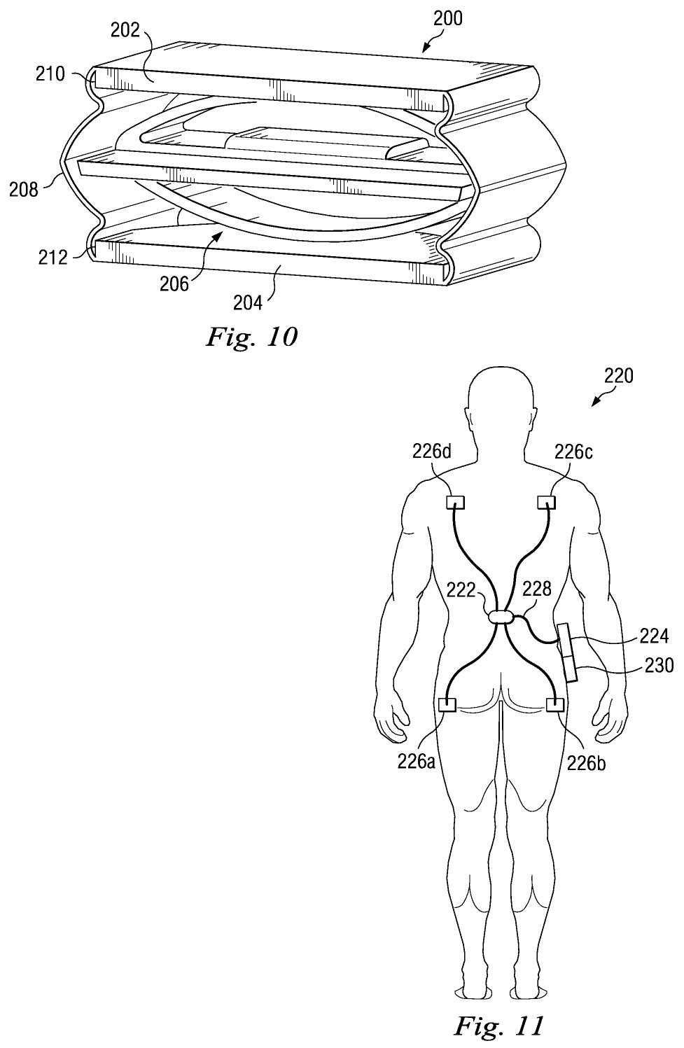

FIG. 10 shows a cross-section of an additional embodiment of an exemplary prosthetic device 200. This embodiment includes upper and lower endplates 202, 204 with a displacement element 206 therebetween. The upper and lower endplates 202, 204 and the displacement element 206 may be configured as described above. In addition, the prosthetic device includes a sheath 208 extending from an edge 210 of the upper endplate 202 to an edge 212 of the lower endplate 204. This sheath 208 may be configured to seal the prosthetic device from body tissue and fluids present at the implantation site. In some embodiments, the prosthetic device 200 may be filled with a fluid prior to implantation, such as a saline or other biocompatible fluid. The sheath 208 may be formed of any biocompatible material, including bionate polyethelene, silicone materials, elastomers, or other flexible materials. In some embodiments, such as the one shown, the sheath extends from endplate edge to endplate edge, while in other embodiments, the entire prosthetic device including the endplates is encased in the sheath. In yet other embodiments, the sheath extends from an inner surface of the upper endplate 202 to an inner surface of the lower endplate 204. The sheath 208 may attach to the upper and lower endplates 202, 204 by any method. For example, it may be attached by bonding or adhering, welding, clamping, among others.

FIG. 11 is an illustration showing one example of a dynamic prosthetic device system 220 implanted in a body. The system 220 includes a prosthetic device 222, a power source 224, and one or more optional environment sensors 226a-d. The prosthetic device 222 is a dynamic prosthetic device and includes displacement elements as described above. It may be any of the exemplary prosthetic devices described above or another similar prosthetic device. Disposed between vertebral bodies in a body's spinal column, the prosthetic device 222 is configured to support and provide functionality to the patient.

The power source 224 may be any device configured to provide power to the displacement elements in the prosthetic device 222. In the embodiment shown, the power source 224 is a battery pack disposed outside the patient's body. However, in other embodiments, the power source 224 is implanted inside the body, and may be implanted inside the prosthetic device 222 itself, such as in the endplates, between the endplates, or otherwise disposed. Thus in some embodiments, the prosthetic device may be internally powered. In some embodiments, the battery power source 224 may be a lithium iodine battery similar to those used for other medical implant devices such as pacemakers. It is fully contemplated that any battery source may be rechargeable. It is also contemplated that when the battery power source is implanted, it may be recharged by an external device so as to avoid the necessity of a surgical procedure to recharge the battery. For example, in one embodiment the battery 150 is rechargeable via inductive coupling.

In yet other embodiments, the prosthetic device may be self-powered, not requiring a separate power supply. For example, a piezoelectric transducer may be utilized such that signals generated by detected by the transducer also provide power to the prosthetic device. The piezoelectric transducers convert energy into an electrical signal that is stored for later use. Accordingly, in some embodiments, such power sources could utilize patient motion to maintain a power supply. In the embodiment shown in FIG. 10, however, a conductor 228 extends from the power source 224 to the prosthetic device 222 and provides power to the displacement elements.

The exemplary system 220 in FIG. 10 includes four optional environment sensors 226a-d in communication with the prosthetic device 222. These sensors may monitor the body's condition or orientation. For example, the sensors may detect or monitor one or more of pressure, temperature, body position, such as sitting or bending, body orientation, acceleration, loads or forces, among others. Data captured by the sensors 226 may be used to adjust the prosthetic device 222 in real time, allowing the prosthetic device to adjust in response to the detected properties or conditions. In some exemplary embodiments, the sensors send electrical current to the prosthetic device 222 to activate the displacement elements. Accordingly, the prosthetic device 222 may adjust or articulate in real time in a manner that mimics a natural physiological disc.

In the embodiment shown, the sensors 226 are disposed at the hips and shoulders. However, the sensors may be disposed at other locations in the body. For example, they may be disposed at the knees, feet, arms or legs. In some embodiments, the sensors are disposed adjacent natural intervertebral prosthetic devices above or below the prosthetic device 222 and provide feedback regarding the real-time characteristics and load carried by the natural discs. This data may be used to adjust the prosthetic device 222 to mimic the natural discs.

In some embodiments, the system 220 includes a controller 230 for processing the data obtained by the sensors 226 and for generating a signal to control the displacement members of the prosthetic device 222 based on the data obtained by the sensors. The controller may be disposed onboard the prosthetic device 202 as described above with reference to FIGS. 4 and 5, or elsewhere in or about the patient's body. In the embodiment shown, the controller is disposed outside the body adjacent the power source 224.

The controller 230 may be configured to receive and process data obtained by the sensors 226a-d, or alternatively, receive and process data entered by a treating physician using any known input device, including a keyboard, hand-operated mouse, or other known devices. Based upon the data received from whichever source, the controller 230 may generate signals that are communicated to and actuate the actuatable displacement elements. So doing allows the controller to control the actuatable displacement elements to change the tilt, pitch, and distance between the upper and lower endplates of the prosthetic device.

In some embodiments, the sensors 226 are in wired communication with the controller 230. In other embodiments, the sensors 226 operate remotely from the prosthetic device 222, the power source 224, and the controller 230. In these embodiments, the sensors 226 may broadcast data using a wired or a wireless system. For example using a wireless system, the sensors may wirelessly communicate with either the prosthetic device 222 or the controller 230, and the controller 230 may wirelessly communicate with the prosthetic device 222. There are several types of wireless systems that may be employed for communication between the sensors 226, the prosthetic device 222, the power source 224, and the controller 230. For example, RFID, inductive telemetry, acoustic energy, near infrared energy, “Bluetooth,” computer networks, among others are all possible means of wireless communication.

In use, a physician may implant the prosthetic device, close the surgical site, and afterward adjust in situ the location, height, orientation, or tilt to best alleviate any pain or distress of the patient. Accordingly, the physician may make post-operative adjustments to the prosthetic device without requiring additional surgery. The physician may then lock the device to create a rigid system. Alternatively, the physician may monitor the system and provide additional adjustment as needed. For example, additional height increases may be needed as a patient grows. In some embodiments, the adjustments can be made using wired or wireless systems. As a further alternative, the prosthetic device may receive data from the sensors and adjust the displacement elements in real time according to the detected needs of the body.

It should be noted that although this disclosure illustrates use within the spinal column, it is fully contemplated that prosthetic devices incorporating the subject matter of this disclosure may be utilized throughout the skeletal system. For example, but without limitation to other applications, in other embodiments the prosthetic device may be used at the knee joint, at the acetabular cup, or at the shoulder.

Generally, the prosthetic device may be implanted into a body using a posterior transforaminal approach similar to the known transforaminal lumbar interbody fusion (TLIF) or posterior lumbar interbody fusion (PLIF) procedures. PLIF approaches are generally more medial and rely on more retraction of the traversing root and dura to access the vertebral interspace. TLIF approaches are typically more oblique, requiring less retraction of the exiting root, and less epidural bleeding with less retraction of the traversing structures. It is also possible to access the interspace using a far lateral approach. In some instances it is possible to access the interspace via the far lateral without resecting the facets. Furthermore, a direct lateral approach through the psoas is known. This approach avoids the posterior neural elements completely. It is anticipated that embodiments of the prosthetic device 100 could utilize any of these common approaches.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

I claim:

1. An active vertebral prosthetic device system, comprising:
an actuatable displacement element having an upper side and a lower side, the actuatable displacement element being configured for placement between an upper vertebral body and a lower vertebral body and being configured to alter the overall distance between the upper and the lower vertebral bodies in situ; and
a controller operable to post-surgically actuate the actuatable displacement element.

2. The active vertebral prosthetic device system of claim 1, comprising:
a first endplate cooperating with the upper side of the actuatable displacement element and being configured to cooperatively engage the upper vertebral body; and
a second endplate cooperating with the lower side of the actuatable displacement element and being configured to cooperatively engage the lower vertebral body.

3. The active vertebral prosthetic device system of claim 2, wherein the actuatable displacement element is an actuator extending between and being in contact with the first and the second endplates.

4. The active vertebral prosthetic device system of claim 2, wherein each of the first and second endplates includes an inner surface, and wherein the actuatable displacement element is disposed at a non-perpendicular angle relative to each of the inner surfaces.

5. The active vertebral prosthetic device system of claim 4, wherein the actuator is aligned relative to the inner surfaces at an angle between 10 and 80 degrees.

6. The active vertebral prosthetic device system of claim 2, wherein the actuatable displacement element is a piston aligned substantially parallel to at least one of the first and second endplates.

7. The active vertebral prosthetic device system of claim 2, wherein the actuatable displacement element is operable to tilt one of the first and second endplates relative to the other of the first and second endplates.

8. The active vertebral prosthetic device system of claim 1, wherein the actuatable displacement element is a piezoelectric motor.

9. The active vertebral prosthetic device system of claim 1, wherein the actuatable displacement element is one of an electroactive polymer and an ionic polymer metal composite.

10. The active vertebral prosthetic device system of claim 1, wherein the actuatable displacement element is a piezoelectric actuator.

11. The active vertebral prosthetic device system of claim 1, wherein the actuatable displacement element comprises at least three piezoelectric motors disposed symmetrically relative to a sagittal plane.

12. The active vertebral prosthetic device system of claim 1, wherein the actuatable displacement element is a first actuatable displacement element, the vertebral prosthetic device system further comprising:
a second actuatable displacement element; and
a connecting rod extending between the first and the second actuatable displacement elements.

13. The active vertebral prosthetic device system of claim 1, wherein the actuatable displacement element is a hexapod.

14. The active vertebral prosthetic device system of claim 1, further including a sheath extending at least partially about the actuatable displacement element.

15. An active vertebral prosthetic device system comprising:
a first endplate configured to cooperatively engage an upper vertebral body;
a second endplate configured to cooperatively engage a lower vertebral body;
an actuatable displacement element operably disposed between the first and the second endplates, the actuatable displacement element being configured to change the overall distance between the first endplate and the second endplate in situ; and
a controller operable to post-surgically actuate the actuatable displacement element.

16. The active vertebral prosthetic device system of claim 15, wherein the displacement element is a piezoelectric motor.

17. The active vertebral prosthetic device system of claim 15, wherein the displacement element is one of an electroactive polymer and an ionic polymer metal composite.

18. The active vertebral prosthetic device system of claim 15, wherein the displacement element is an actuator.

19. The active vertebral prosthetic device system of claim 15, comprising:
upper vertebral body attachment features cooperatively associated with the first endplate and being configured to engage the upper vertebral body; and
lower vertebral attachment features cooperatively associated with the second endplate and being configured to engage the lower vertebral body.

20. A vertebral prosthetic device system for implantation in a body, comprising:
an active vertebral prosthetic device including an actuatable displacement element having an upper side and a lower side, the active vertebral prosthetic device being configured for placement between and in contact with an upper vertebral body and a lower vertebral body and being configured to alter the overall distance between the upper and the lower vertebral bodies in situ;
a controller operable to post-surgically communicate with the active vertebral prosthetic device; and
a sensor configured for implantation in the body, the sensor being in communication with the controller and being configured to provide data to the controller, wherein the controller is configured to process the data and control the actuatable displacement element.

21. The vertebral prosthetic device system of claim 20, wherein the active vertebral prosthetic device comprises:
a first endplate disposed at the upper side of the actuatable displacement element and being configured to cooperatively engage the upper vertebral body; and
a second endplate disposed at the lower side of the actuatable displacement element and being configured to cooperatively engage the lower vertebral body.

22. The vertebral prosthetic device system of claim 20, further comprising a power source associated with the actuatable displacement element.

23. The vertebral prosthetic device system of claim 20, wherein the actuatable displacement element is a piezoelectric motor.

24. The vertebral prosthetic device system of claim 20, wherein the actuatable displacement element is one of an electroactive polymer and an ionic polymer metal composite.

25. The vertebral prosthetic device system of claim 20, wherein the actuatable displacement element is an actuator.

26. The vertebral prosthetic device system of claim 20, wherein the controller is configured to process the data provided by the sensor to control the actuatable displacement element in real time.

27. A method comprising:
implanting an actuatable displacement element between an upper vertebral body and a lower vertebral body, the actuatable displacement element having an upper side and a lower side respectively facing the upper vertebral body and the lower vertebral body; and
post-surgically actuating the actuatable displacement element with a controller to alter the overall distance between the upper and lower vertebral bodies.

28. The method of claim 27, wherein the implanting of the actuatable displacement element includes:
cooperatively engaging a first endplate with the upper vertebral body; and
cooperatively engaging a second endplate with the lower vertebral body, wherein the actuatable displacement element is disposed between and cooperates with the first and the second endplates.

29. The method of claim 27, further comprising providing power to the displacement element with a power source.

30. The method of claim 27, further comprising processing data from a sensor, wherein the post-surgically actuating of the actuatable displacement element is based on the data.

31. The method of claim 27, wherein the displacement element is a piezoelectric motor.

32. The method of claim 27, wherein the displacement element is one of an electroactive polymer and an ionic polymer metal composite.

33. The method of claim 27, wherein the displacement element is an actuator.
